(19) United States
(12) Patent Application Publication
(10) Pub. No.: US 2019/0361001 A1
(43) Pub. Date: Nov. 28, 2019

(54) MOLYBDENUM TRIOXIDE AND NANO SILICON CHIPS FOR ACETONE DETECTION

(71) Applicant: INDIAN INSTITUTE OF TECHNOLOGY DELHI, Delhi (IN)

(72) Inventors: Saakshi DHANEKAR, Delhi (IN); Priyanka DWIVEDI, Delhi (IN); Samaresh DAS, Delhi (IN)

(21) Appl. No.: 16/479,763

(22) PCT Filed: Sep. 22, 2017
(86) PCT No.: PCT/IN2017/050421

§ 371 Date: Jul. 22, 2019

(30) Foreign Application Priority Data
Jan. 20, 2017 (IN) 201711002327

(51) Int. Cl. G01N 33/497; G01N 27/12; C25D 11/24; C25D 11/32; C23C 14/08; C23C 14/58; C23C 14/34

(52) U.S. Cl. CPC G01N 33/497; G01N 27/127; C25D 11/24; G01N 2033/4975; C23C 14/083; C23C 14/5853; C23C 14/34; C25D 11/32

(57) ABSTRACT

The present invention relates to a sensor chip and more particularly to the sensor chip for detection of acetone. In one embodiment, the sensor chip comprising: a first layer having a nano-porous silicon fabricated on a P-type Si <100> substrate, a second layer having molybdenum trioxide (MoO₃) in contact with the first layer and a third layer having chrome gold inter digitated electrodes in contact with the second layer.

### FIELD OF THE INVENTION

The present invention mainly relates to a sensor chip and more particularly to the sensor chip for detection of acetone.

### BACKGROUND OF THE INVENTION

Sensors are well known in the art which is a device that measures a physical quantity and converts it into a ‘signal’ which can be read by an observer or by an instrument. Nowadays, demand for sensors used in medical diagnostics and in other fields has been increased, and further, development of a sensor and a detection device small in size and capable of performing sensing at high speed has been demanded. In order to meet such demand, a variety of types of sensors such as a sensor using different types of electrochemical process have been studied.

In subjects suffering from diabetes, the body does not make much insulin, but cells still need glucose as a fuel. To compensate for the lack of insulin, cells burn fat in which process, produces ketones (acetone). When these ketones rise to unsafe levels, the condition is called diabetic ketoacidosis (KDA). Thus, the ketones measurement/monitoring is one of an alternative to the traditional glucose monitoring of diabetic patients by using blood tests. The conventional testing requires blood draws, which is cumbersome, discomfort to the patient, requires trained personnel, and is not easy to implement in rural settings where access to healthcare is minimal.

Nowadays, the selective detection of volatile organic compounds (VOCs) is utilized in a number of settings such as monitoring air quality in polluted environments, monitoring emissions in factory or vehicle exhausts and the like. More recently, it has been recognized that exhaled human breath contains volatile organic compounds such as ethanol or acetone etc. which can be indicators of human health. Specifically, it has been recognized that the levels of acetone in exhaled human breath can be a diagnostic tool for providing information on the status of patients suffering from diabetes. The acetone concentration in human breath varies from 300 to 900 ppb in healthy people to more than 1800 ppb for diabetics.

Noninvasive detection of diseases by breath analysis is fast, and provides field deployable economically viable alternatives to blood analysis and/or diagnostic tests like endoscopy. A number of previously described sensors for detection of volatile organic compounds require high sensor operating temperatures, typically in ranges of 200° C. to 500° C. For example, sensors comprising tungsten doped silicon have been described for selective detection of acetone wherein the Si:WO₃ sensor film is heated to about 350° C. during operation of the device. Such high operating temperatures are not easily achievable in non-laboratory settings. While certain sensors which are operable at room temperature have been described for detection of VOCs (e.g., sensors based on nano-porous silicon), such sensors suffer from drawbacks because they are not selective and often readout false positives because they are sensitive to the moisture present in samples.

In addition to acetone, there are low levels of other volatile organic compounds such as methanol or ethanol in human breath. Thus any sensor for detection of acetone in human breath needs to be selective for acetone while remaining less sensitive or not sensitive to humidity and/or other volatile organic compounds.

Therefore there is a need in the art with the diagnostic testing tool (sensor chip) having high sensitivity and selectivity for detection of acetone and to solve the above mentioned limitations.

### SUMMARY OF THE INVENTION

An aspect of the present invention is to address at least the above-mentioned problems and/or disadvantages and to provide at least the advantages described below.

Accordingly, in one aspect of the present invention relates to a sensor chip for selective detection of acetone, the sensor chip comprising: a first layer having a nano-porous silicon fabricated on a P-type Si <100> substrate, a second layer having molybdenum trioxide (MoO₃) in contact with the first layer and a third layer having chrome gold inter digitated electrodes in contact with the second layer.

In another aspect of the present invention relates to a method for fabricating a sensor chip for selective detection of acetone at room temperature, the method comprising: anodizing P-type Si <100> substrate in an electrolytic solution having hydrofluoric acid and ethanol, fabricating silicon nanowires by metal assisted chemical etching of silicon in hydrofluoric acid solution, providing a MoO₃ thin film on the substrate of step (a) by depositing molybdenum thin film on the substrate of step (a) by radio frequency reactive sputtering, oxidizing molybdenum thin film in a furnace, and depositing inter digitated electrodes (IDEs) on the MoO₃ film by a shadow mask in sputtering.

In another aspect of the present invention relates to a breath analyzer device for detecting acetone comprising: a sensor chip for selective detection of acetone at room temperature, wherein the sensor chip including: a first layer having a nano-porous silicon fabricated on a P-type Si <100> substrate, a second layer having molybdenum trioxide (MoO₃) in contact with the first layer, third layer having chrome gold inter digitated electrodes in contact with the second layer and a sensor header and an integrated microcontroller.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE DRAWINGS

The above and other aspects, features, and advantages of certain exemplary embodiments of the present invention will be more apparent from the following description taken in conjunction with the accompanying drawings in which:

FIG. 1 shows the schematic diagram of an electrochemical cell for nano-porous silicon fabrication according to one embodiment of the present invention.

FIG. 2 shows the process flow for fabrication of MoO₃ deposited on a nano-porous silicon substrate for incorporation in a sensor/chip/wafer for detection of acetone and includes step A of FIG. 1 and step B (oxidation) and step C (IDE fabrication) according to one embodiment of the present invention.

FIG. 3 shows IDEs deposited on MoO₃ and nano-porous silicon sensor/chip/wafer according to one embodiment of the present invention.

FIG. 4(i) shows FESEM micrographs (a) MoO₃ assembled NPS samples (b) magnified view of MoO₃ assembled NPS samples (c) NPS morphology under MoO₃ (d) cross-sectional SEM image of MoO₃ assembled NPS (e) magnified view of top NPS surface showing porous network according to one embodiment of the present invention.

FIG. 4(ii) shows FESEM micrographs of MoO3 assembled silicon nanowires (Si nW) samples.

FIG. 5 shows XRD of three different samples: Mo deposited on NPS prepared from p-type silicon, MoO₃ deposited on NPS prepared from p-type silicon, and MoO₃ deposited on crystalline p-type Si according to one embodiment of the present invention.

FIG. 6 shows results from sensor studies of a sensor comprising nano-porous silicon having MoO₃ deposited thereon in presence of different analytes, namely ethanol, isopropyl alcohol and acetone according to one embodiment of the present invention.

FIG. 7(a) shows sensor response in 90% RH to different concentrations of acetone (with error bars), and (b) Dynamic response of sensor to wide range of ppm acetone (0.5 to 100 ppm) according to one embodiment of the present invention.

FIG. 8 shows sensor response percent of nano-porous silicon to varying concentrations of acetone according to one embodiment of the present invention.

FIG. 9 shows results from sensor studies of a sensor comprising nano-silicon (silicon nanowire) having MoO₃ deposited thereon in presence of varying concentration of acetone according to one embodiment of the present invention.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure.

Throughout the drawings, it should be noted that like reference numbers are used to depict the same or similar elements, features, and structures.

### DETAILED DESCRIPTION OF THE INVENTION

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

It is to be understood that the singular forms ‘a, ‘an, and ‘the include plural referents unless the context clearly dictates otherwise. Thus, for example, reference to ‘a component surface includes reference to one or more of such surfaces.

By the term ‘substantially it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

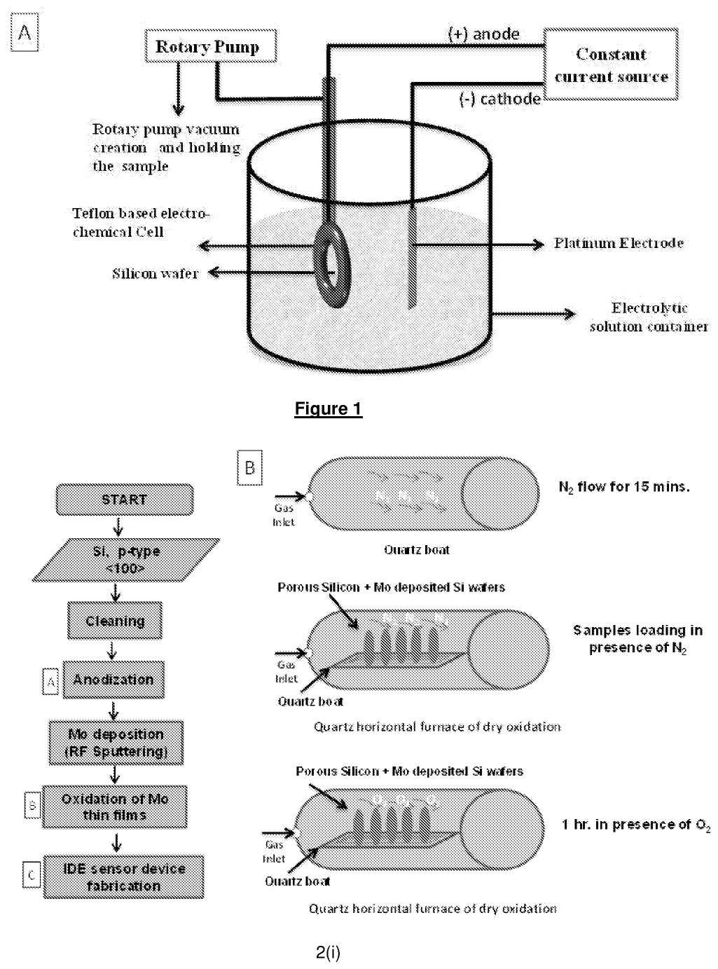

FIGS. 1 through 9, discussed below, and the various embodiments used to describe the principles of the present disclosure in this patent document are by way of illustration only and should not be construed in any way that would limit the scope of the disclosure. Those skilled in the art will understand that the principles of the present disclosure may be implemented in any suitably arranged communications system. The terms used to describe various embodiments are exemplary. It should be understood that these are provided to merely aid the understanding of the description, and that their use and definitions in no way limit the scope of the invention. Terms first, second, and the like are used to differentiate between objects having the same terminology and are in no way intended to represent a chronological order, unless where explicitly stated otherwise. A set is defined as a non-empty set including at least one element.

The present invention is a chip or chip-based sensors comprising MoO₃ layered on nano silicon which are operable at room temperature for selective detection of acetone at sub-ppm levels and which can be used for sensing acetone levels in the breath of a patient. The measured acetone levels in the breath of a patient can in turn be correlated with sugar levels/diabetic status in the human body. The chip fabrication process described herein is reproducible, scalable to wafer scale, and compatible with Integrated Circuit (IC) industry processes. The chips provided herein can be packaged into a sensor header and integrated with a microcontroller to provide a handheld device. Accordingly, provided herein are portable devices for monitoring acetone levels in the exhaled breath of patients (e.g., diabetics). Provided herein is a chip for selective detection of acetone, the chip comprising: a first layer comprising nano-silicon, a second layer comprising molybdenum trioxide (MoO₃) in contact with the first layer and inter digitated electrodes (IDE).

In embodiments of the chip/sensor described herein, the detection of acetone occurs at room temperature, without the need for raising the temperature of the sensor to high temperatures (e.g., 100-400° C. and the like.)

Accordingly, provided herein are sensors for selective detection of acetone, including selective detection of acetone in the presence of other VOCs and moisture. The sensors described herein comprise nano-silicon and molybdenum trioxide (MoO₃). Advantageously the chips and/or sensors described herein are operable at room temperature and are easily deployable in the field.

Manufacturing of other metal-oxide based sensors which operate at high temperatures presents challenges during packaging, device integration and device operation. Various processes used for fabrication of metal oxide nanostructures such as sol-gel techniques and other wet techniques are not scalable and do not provide uniformity of structures on chips. An advantage of the chips described herein is that they are suitable for wafer scale fabrication because the fabrication of the chips described herein employs techniques such as radio frequency sputtering or radio frequency reactive sputtering thereby allowing for reproducible and scalable fabrication along with uniformity in structures on chips. A large number of devices can be accommodated in a smaller region thereby reducing the manufacturing costs of the wafer/chip. Further, the chips described herein are usable at room temperature and the same chip can be used multiple times.

The chips described herein can selectively detect acetone even under conditions of 90% relative humidity (RH) and/or in the presence of other organic compounds such as ethanol and isopropyl alcohol. The chips described herein can be packaged into a sensor header and integrated with a microcontroller to make a portable handheld device. Accordingly provided herein is a portable handheld device which can be used for glucose monitoring, wherein the device detects/measures acetone at room temperature without the need for heating the chip/sensor to high temperatures, and wherein the device comprises chips/sensors comprising nano-silicon and molybdenum trioxide as described herein.

Although other metal-oxide-plus-silicon chips/sensors are known, the forms of silicon employed in previously described chips and/or the metal oxides employed in such chips require higher temperatures for operation and/or detection of the presence of VOCs. By contrast the chips described herein employ nano-silicon (e.g., nano-porous silicon, nanocrystalline silicon, semi-crystalline silicon, nanowires/nanotubes) which, in combination with molybdenum trioxide, results in formation of hetero junctions comprising p-type silicon and n-type molybdenum trioxide, unexpectedly allowing for operation of the chips/sensors at room temperature. The chips/devices provided herein are easy to use for glucose monitoring compared to the conventional finger prick blood draw methods used traditionally for diabetic patients.

As used herein ‘nano-silicon refers to a range of silicon materials embedded in a porous matrix. In some embodiments nano-silicon is nano-porous silicon, having a web-like structure comprising pores and silicon nano-crystals embedded in a porous matrix. In other embodiments, nano-silicon refers to silicon nanowires in a bunched/bundled form. Nano-silicon may comprise one or more of nano-porous silicon, nanocrystalline silicon, semi-crystalline silicon, nanowires/nanotubes and the like.

As used herein, ‘wafer scale or ‘wafer scale fabrication refers to the ability to reliably and reproducibly manufacture chips/sensors with minimal variability in the detection capability of the chip/sensor. Typically, wafer diameters used during etching/fabrication range from 25 mm to 205 mm or up to about 8 inches. The thickness of a wafer may vary depending on the diameter.

As used herein ‘P-type <100> Si substrate refers to a silicon substrate (e.g.

wafer) having boron as dopant

As used herein, ‘anodization of silicon substrate refers to introduction of pores in the silicon substrate through the use of an anodization cell.

As used herein, ‘room temperature is ambient temperature and may vary but is typically in a range of 20° C. to 30° C. However it will be understood that ‘room temperature may vary by geographic regions and all such equivalent ‘room temperatures are contemplated within the scope of embodiments described herein.

As used herein, MoO₃⁻ deposited/layered nano silicon (or MoO₃ deposited/layered nano-porous silicon, or MoO₃ deposited/layered silicon nanowire) refers to a first silicon layer in contact with a second layer which is a MoO₃ layer as described herein in the examples section.

Described herein is a scalable fabrication process for MoO₃ deposited/layered nano-porous silicon, and chips/sensors/wafers thereof. Nano porous silicon (NPS) samples fabricated using anodization technique on P-type Si <100> substrate in a mixed electrolytic solution of hydrofluoric acid and ethanol. Some of the parameters which affect the morphology of NPS are electrolytic solution composition, current density and etching time. Etching time can vary from 30 secs to 60 mins. Current density can vary from 1 mA/cm² to 100 mA/cm². For P-type silicon, etching is carried out using hydrofluoric acid and ethanol in ratios of 1-4:4-1 (e.g., a hydrofluoric acid:ethanol ratio of 1:1, 1:2, 1:3, 1:4, 2:1, 3:1; 4:1. Silicon nanowire is fabricated using metal (gold nanodots, silver nanodots) assisted chemical etching in hydrofluoric acid based solutions. The Mo thin film is deposited using reactive radio frequency (RF) sputtering, and then the thin film is oxidized in a furnace. All the steps described herein are scalable, reproducible, and allow for wafer scale fabrication of chips/sensors.

FIG. 1 shows the schematic diagram of an electrochemical cell for nano-porous silicon fabrication according to one embodiment of the present invention.

The figure shows the schematic diagram of an electrochemical cell for nano-porous silicon fabrication. Nano-porous silicon (NPS) samples are fabricated using anodization technique on P-type Si <100> substrate with resistivity 1-10 ‘n-cm in an electrolyte solution of HF:C₂H₅OH in 1:1 ratio using electrochemical cell. FIG. 1 shows the schematic diagram of in house-made electrochemical cell for NPS fabrication process. Some of the parameters which affect the morphology of NPS A are electrolytic solution composition, current density and etching time. Samples i.e. nano-porous silicon is fabricated at current density of 50 mAcm⁻² etching for a duration of 15 minutes and the etched substrate is used as a platform for MoO₃ nanostructures growth.

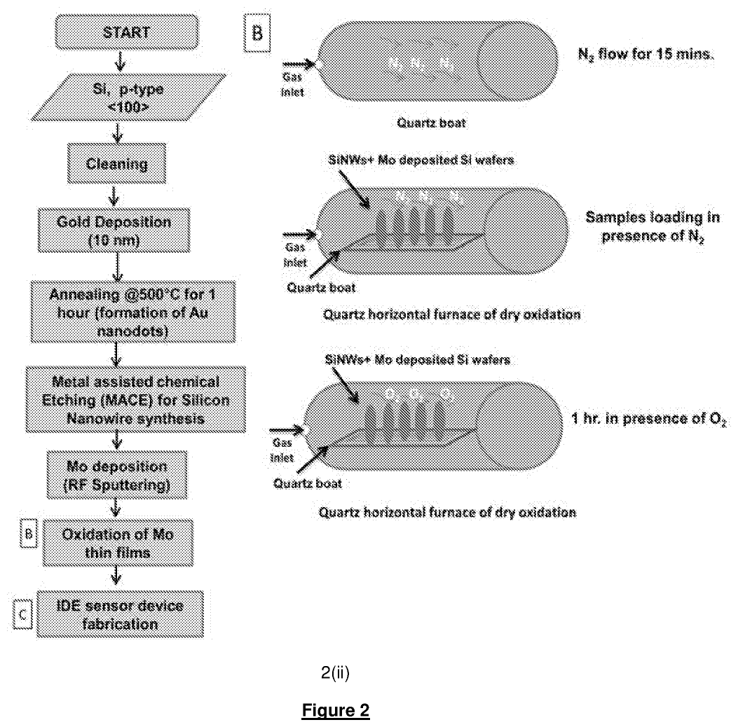

FIG. 2 shows the process flow for fabrication of MoO₃ deposited on a nano-porous silicon substrate for incorporation in a sensor/chip/wafer for detection of acetone and includes step A of FIG. 1 and step B (oxidation) and step C (IDE fabrication) according to one embodiment of the present invention.

The figure shows the process flow for fabrication of MoO₃ deposited on a nano-porous silicon substrate for incorporation in a sensor/chip/wafer for detection of acetone and includes step A of FIG. 1 and step B (oxidation) and step C (IDE fabrication). A Molybdenum (Mo) target (purity 99.999%) of 75 mm diameter is used to deposit Mo thin films using RF sputtering. Mo film of 10 nm is deposited at 100 W RF power, 10 mtorr gas pressure and keeping the substrate to target distance as 65 mm with no substrate heating. Mo thin film thickness and uniformity are checked using stylus surface profiler (KLA Tencor, Alpha step IQ) by making a step along the diameter of wafer. Oxidation of the deposited molybdenum film is conducted in a quartz horizontal furnace which is fitted with a rotameter to control the flow of oxygen (as shown in FIG. 2). In the oxidation furnace, initially oxygen ambient is established by maintaining a flow rate of 1 liter per minute through the rotameter. Then, Mo deposited Si wafers are placed in the centre zone of the furnace and nitrogen is introduced inside the furnace for 10 minutes. Following this, dry oxidation is performed in oxygen ambient at 500 éC temperatures.

FIG. 2(ii) describes initially, a gold film of 10 nm is deposited on a silicon wafer and annealed to form gold nanodots. Then, the wafer undergoes a metal assisted chemical etching (MACE) in presence of HF: H₂O₂:C₂H₅OH (5:1:1) solution with time duration ranging from 2 to 30 minutes. This leads to the formation of silicon nanowire. The wafer is dipped in gold etchant to remove the excess gold from the wafer. Molybdenum (Mo) (˜10-30 nm) is deposited using RF sputtering after formation of nanowires, is oxidized @500 eĆfor 60 minutes. Chrome-gold are then deposited using sputtering and metal mask for formation of electrodes on the surface.

The present invention relates to a sensor chip for selective detection of acetone, the sensor chip comprising: a substrate which is a p-type silicon, a first layer comprising nano-porous silicon, a second layer comprising molybdenum trioxide (MoO₃) in contact with the first layer, a third layer comprising of chrome gold inter digitated electrodes in contact with second layer.

The nano-porous silicon is fabricated using anodization technique on P-type Si <100> substrate with resistivity 1-10 ‘n-cm in an electrolyte solution of HF:C₂H₅OH in 1:1 ratio using electrochemical cell. The molybdenum trioxide is fabricated using oxidation of molybdenum thin film. The chrome gold inter digitated electrodes deposited over the MoO₃ assembled NPS film using metal mask in RF sputtering.

The first layer comprising nano-silicon comprises nano-porous silicon (NPS), which is a web like structure containing pores and silicon crystals in nano-dimension where the crystals are embedded in a porous matrix. In another embodiment, the first layer comprising nano-silicon comprises silicon nanowires. The first layer comprising nano-silicon has a thickness ranging from about 500 nm to about 10 microns. In another embodiment, the first layer comprising nano-silicon has a thickness of about 1-5 microns. In some embodiments, the first layer comprising nano-silicon has a thickness of about 5 microns.

In one group of embodiments, the second layer comprising MoO₃ has a thickness ranging from about 1 nm to about 30 nm. In some embodiments, the second layer comprising MoO₃ has a thickness of about 10 nm.

In one group of embodiments, the detection range for acetone ranges from about 0.5 ppm to about 100 ppm.

In some of such embodiments, the chip is selective for detection of acetone over isopropanol and ethanol in the presence of up to 90% relative humidity (RH). In further embodiments, the nano-silicon substrates having MoO₃ deposited thereon form chips/sensors which are more sensitive to acetone compared to nano-silicon alone.

Also provided herein is a process/method for fabricating a chip for selective detection of acetone at room temperature, the method comprising: anodizing P-type Si <100> substrate in an electrolytic solution having hydrofluoric acid and ethanol, fabricating silicon nanowires by metal assisted chemical etching of silicon in hydrofluoric acid solution, providing a MoO₃ thin film on the substrate of step (a) by depositing molybdenum thin film on the substrate of step (a) by radio frequency reactive sputtering, oxidizing the molybdenum thin film and depositing inter digitated electrodes (IDEs) on the MoO₃ film by a shadow mask in sputtering.

In alternate embodiments, other techniques such as thermal evaporation and atomic layer deposition (ALD) may be used for deposition of molybdenum and are contemplated as being within the scope of embodiments described herein.

In some embodiments, the ratio of hydrofluoric acid: ethanol in step a) is 1-4:4-1. In some embodiments, the ratio of hydrofluoric acid: ethanol in step a) is 1:1 and the silicon substrate is etched with said hydrofluoric acid ethanol mixture for a period ranging from 30 seconds to 60 minutes.

In one group of embodiments the process of fabrication described herein is conducted on wafer scale.

In another aspect, of the present invention relates to a breath analyzer device for detecting acetone comprising: a sensor chip for selective detection of acetone at room temperature, wherein the sensor chip comprises: a substrate which is a p-type silicon, a first layer comprising nano-porous silicon, a second layer comprising molybdenum trioxide (MoO₃) in contact with the first layer, a third layer comprising of chrome gold inter digitated electrodes in contact with second layer.

In some embodiments, the device is a portable device for detection of diabetes by analyzing the exhaled breath of a subject.

In a further aspect, provided herein is a method for detecting diabetes in a human subject, the method comprising: collecting exhaled breath of said human subject in a breath analyzer, where the breath analyzer comprises a sensor chip for selective detection of acetone at room temperature, wherein the sensor chip comprises: a substrate which is a p-type silicon, a first layer comprising nano-porous silicon, wherein the nano-porous silicon is fabricated using anodization technique on P-type Si <100> substrate with resistivity 1-10 ‘n-cm in an electrolyte solution of HF:C₂H₅OH in 1:1 ratio using electrochemical cell, a second layer comprising molybdenum trioxide (MoO₃) in contact with the first layer, wherein the molybdenum trioxide is fabricated using oxidation of molybdenum thin film, a third layer comprising of chrome gold inter digitated electrodes in contact with second layer, wherein the chrome gold inter digitated electrodes deposited over the MoO₃ assembled NPS film using metal mask in RF sputtering, a sensor header and an integrated microcontroller and measuring the level of acetone in the exhaled breath of said subject.

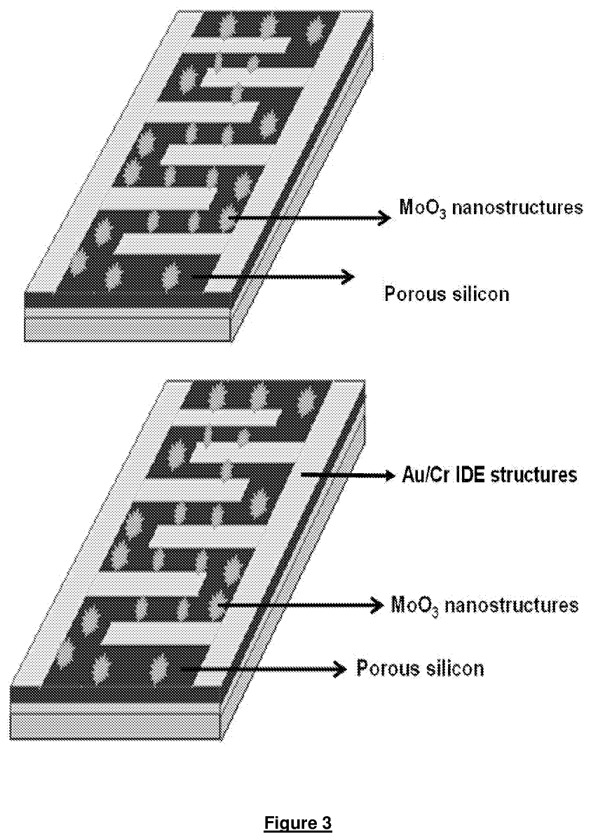

FIG. 3 shows IDEs deposited on MoO₃ and nano-porous silicon sensor/chip/wafer according to one embodiment of the present invention.

The figure shows IDEs deposited on MoO₃ and nano-porous silicon sensor/chip/wafer. The sensor fabrication is completed by depositing chrome gold IDE structures on the MoO₃ assembled NPS samples using metal mask in RF sputtering. The chrome and gold deposition is done using RF sputtering at 300 W power and gas pressure of 20 mtorr and 10 mtorr respectively. The electrodes are made in the form of IDE (inter-digitated electrodes) as shown in FIG. 3. The sensor is tested in the presence of different VOCs and 90% RH to simulate human breath.

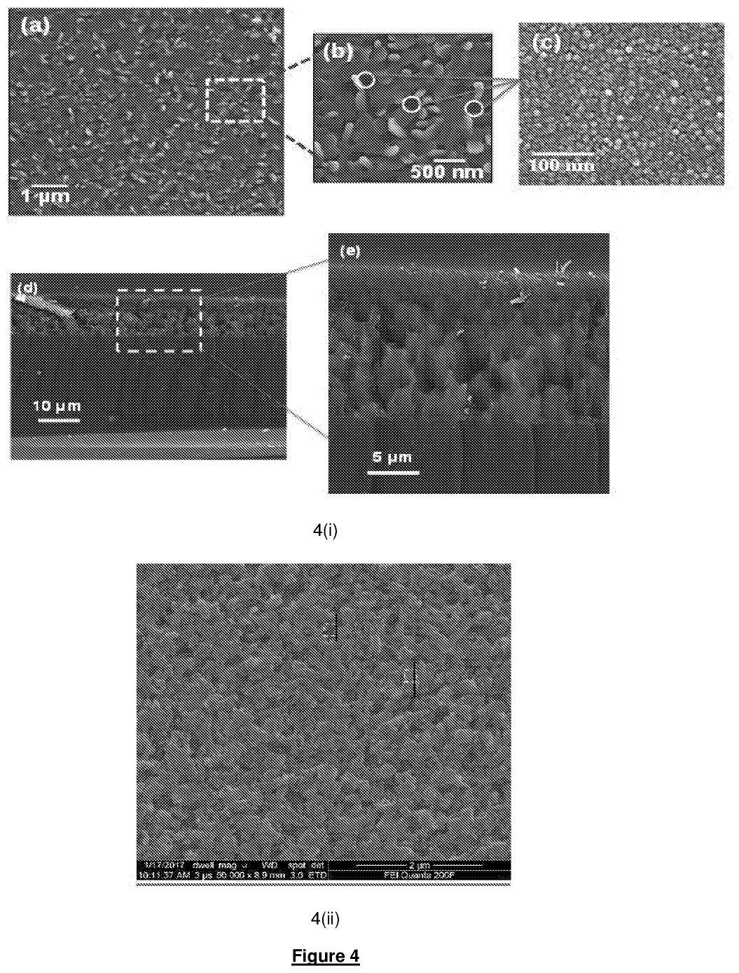

FIG. 4 shows FESEM micrographs (a) MoO₃ assembled NPS samples (b) magnified view of MoO₃ assembled NPS samples (c) NPS morphology under MoO₃ (d) cross-sectional SEM image of MoO₃ assembled NPS (e) magnified view of top NPS surface showing porous network according to one embodiment of the present invention.

The figure shows FESEM micrographs (a) MoO₃ assembled NPS samples (b) magnified view of MoO₃ assembled NPS samples (c) NPS morphology under MoO₃ (d) cross-sectional SEM image of MoO₃ assembled NPS (e) magnified view of top NPS surface showing porous network. As shown in FIG. 4, the morphologies of NPS and MoO₃ assembled NPS samples are examined by SEM (Jeol J S M-7600F and Zeiss EVO 50). Structural analysis is done using Raman spectroscopy using LAbRAMHR Evolution RAMAN Spectrometer (Horiba) with Ar laser excitation wavelength 514 nm. The crystal structure of MoO₃ and MoO₃ assembled NPS samples are analyzed by XRD (Phillips X Pert, PRO-PW 3040 diffractometer) with glancing angle 0.5é.

FIG. 4(a) (b) and (c) depicts the SEM images of MoO₃ assembled NPS samples. The morphology of the NPS samples changes due to assembling of 10 nm Mo thin films after oxidation (FIG. 4(a)). SEM study shows the formation of MoO₃ flakes on NPS sample after performing analysis at higher magnification as shown in FIG. 4(b). FIG. 4(c) shows the morphology of NPS surface which is below the nano-flakes. FIG. 4 (d) and FIG. 4(e) shows the cross-sectional micrographs of MoO₃⁻ nano-sample. The image depicts the formation of NPS pores after MoO₃ deposition. This result in enhanced surface area due to the presence of a combination of NPS and MoO₃ nano flakes compared to the pure NPS sample.

FIG. 4(ii) shows the SEM image and confirmation of formation of MoO₃ flakes on nanowires.

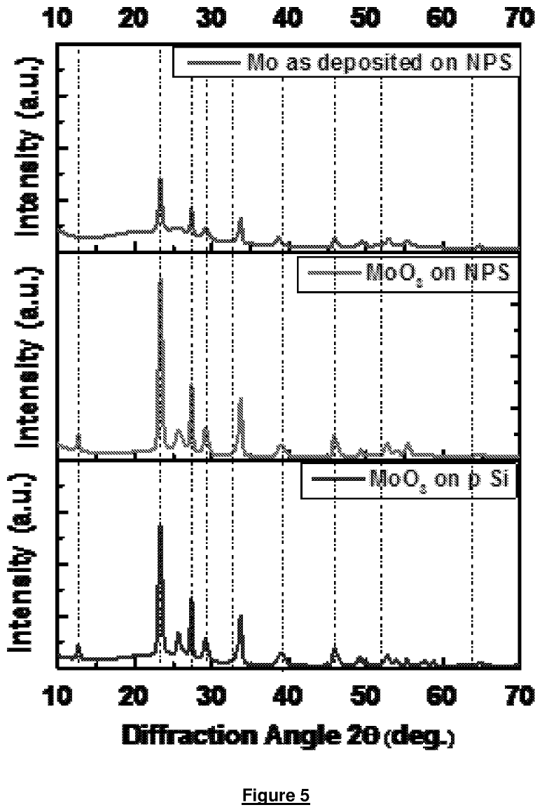

FIG. 5 shows XRD of three different samples: Mo deposited on NPS prepared from p-type silicon, MoO₃ deposited on NPS prepared from p-type silicon, and MoO₃ deposited on crystalline p-type Si according to one embodiment of the present invention.

The figure shows XRD of three different samples: Mo deposited on NPS prepared from p-type silicon, MoO₃ deposited on NPS prepared from p-type silicon, and MoO₃ deposited on crystalline p-type Si. In case of deposited Mo on NPS, due to lack of crystallinity no significant peaks observed for the MoO₃ phase. However, in the case of MoO₃ obtained after oxidation of Mo on p silicon and NPS at 500 éC, peaks corresponding to stable MoO₃ phases are observed. This confirms the formation of poly crystalline and stable MoO₃ with orthorhombic phase. All the diffraction patterns are consistent with orthorhombic MoO₃ having lattice constants a=3.96i, b=13.86 i, and c=3.70 i (JCPDS card no. 75-0192). Samples with different substrates had shown close proximity of peak positions for NPS and p silicon respectively. Orthorhombic MoO₃ (020), (110), (040), (411) and (060) peaks as depicted in FIG. 5 are consistent with literature values Y. Chen et al., C. Lu, L. Xu, Y. Ma, W. Hou, J .-J . Zhu, Cryst. Eng. Comm. 2010, 12, 3740.

### Gas Sensor Testing

Sensing studies were performed by checking change in resistance in the presence of different analytes. The change in resistance is monitored using a Keithley Electrometer 6514 interfaced with a data acquisition system. P. Dwivedi et al., Journal of Materials Science and Technology (2016), vol. 33, 2016, pp. 516-522. Nitrogen gas is used as a carrier gas and the flow rate is controlled by a mass flow controller (MFC). The base value of resistance Rₐ is taken in a compressed air environment and R_{g} is the resistance in the presence of analyte vapors. Sensor response (S), of a sensor is defined as the ratio of change in resistance in presence of test analytes ∀R=R_{g}6Rₐ, to the value of base resistance in compressed air. The sensor response percent by an MoO₃/NPS sensor in the presence of acetone is given by, S=(R_{g}−Rₐ)/Rₐ*100%.

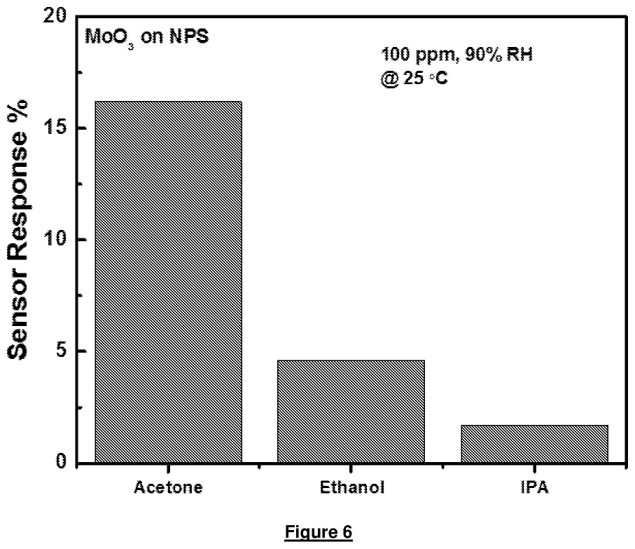

FIG. 6 shows results from sensor studies of a sensor comprising nano-porous silicon having MoO₃ deposited thereon in presence of different analytes, namely ethanol, isopropyl alcohol and acetone according to one embodiment of the present invention.

The figure shows results from sensor studies of a sensor comprising nano-porous silicon having MoO₃ deposited thereon in presence of different analytes, namely ethanol, isopropyl alcohol and acetone. The figure also shows the selectivity to acetone depicted by the sensor described above when checked in 90% RH environment and in the presence of other volatile organic compounds (VOCs).

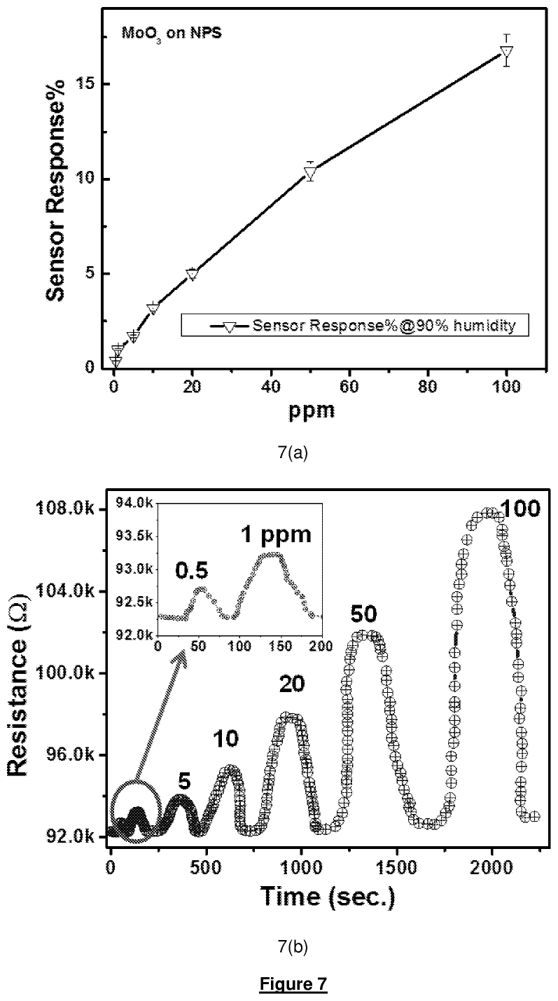

FIG. 7(a) shows sensor response in 90% RH to different concentrations of acetone (with error bars), and (b) Dynamic response of sensor to wide range of ppm acetone (0.5 to 100 ppm) according to one embodiment of the present invention.

The figure shows sensor response in 90% RH to different concentrations of acetone (with error bars), and (b) Dynamic response of sensor to wide range of ppm acetone (0.5 to 100 ppm). The sensor response % to various concentrations of acetone is shown in FIG. 7(a). The graph shows almost linear behavior for a wide concentration range. FIG. 7(b) displays the dynamic response of the sensor to acetone vapor going from 0.5 to 100 ppm concentration. To infer the effect of nano-porous silicon substrate, a few other samples are also tested in the presence of acetone. These are pure MoO₃ (10 nm) and pure nano-silicon.

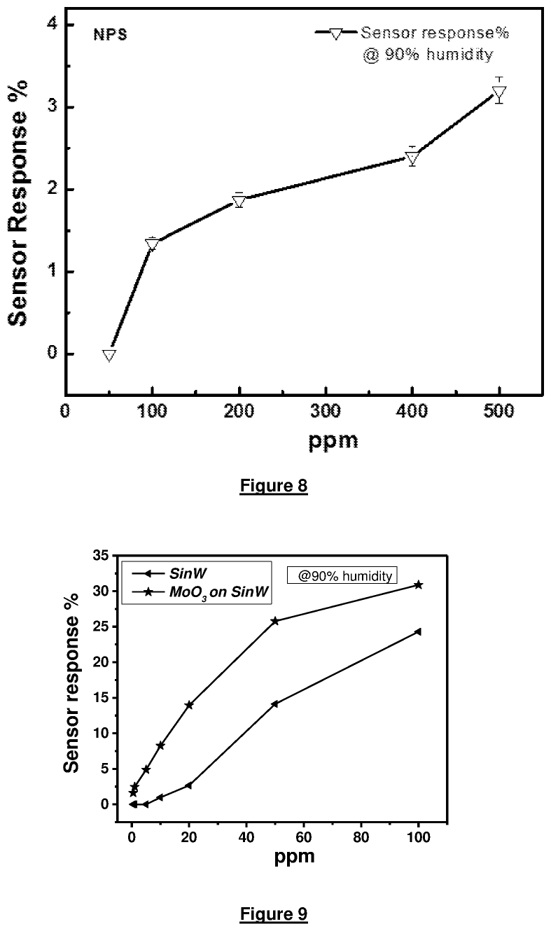

FIG. 8 shows sensor response percent of nano-porous silicon to varying concentrations of acetone according to one embodiment of the present invention.

The figure shows sensor response percent of nano-porous silicon to varying concentrations of acetone. As used herein, the sensor response percent (or sensor response %) refers to the change in sensor response upon exposure to a fixed concentration of analyte. If Ra is the resistance in air, Rg is resistance in the presence of gas, then sensor response % is=[(Rg−Ra)/Ra]×100.

In the former, no response is observed at room temperature as the thickness of the MoO₃ thin film is insufficient, and in the latter case, a response to acetone is observed (FIG. 8). The chip comprising nano-silicon alone is less responsive to acetone at room temperature (FIG. 8) when compared with the response of the MoO₃-nano-silicon chip to acetone at room temperature (FIG. 7(a)). Another version of chip which had silicon nanowire alone and with MoO₃ on top is checked in presence of acetone. The results confirm the enhancement in sensitivity due to the presence of MoO₃ layer.

The chip is selective for detection of acetone over isopropanol and ethanol in the presence of up to 90% relative humidity.

The FIG. 9 shows results from sensor studies of a sensor comprising nano-silicon (silicon nanowire) having MoO₃ deposited thereon in presence of different analytes, namely ethanol, isopropyl alcohol and acetone. This figure shows that the sensor response increased upon addition of MoO₃ and the limit of detection improved with significant change being observed at lower ppm of acetone.

Although the invention has been described in terms of preferred embodiment, it is not limited thereto. Those skilled in this technology can make various alterations and modifications without departing from the scope and spirit of the invention. Therefore, the scope of the invention shall be defined and protected by the following claims and their equivalents.

FIGS. 1-9 are merely representational and are not drawn to scale. Certain portions thereof may be exaggerated, while others may be minimized. FIGS. 1-9 illustrate various embodiments of the invention that can be understood and appropriately carried out by those of ordinary skill in the art.

In the foregoing detailed description of embodiments of the invention, various features are grouped together in a single embodiment for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus, the following claims are hereby incorporated into the detailed description of embodiments of the invention, with each claim standing on its own as a separate embodiment.

It is understood that the above description is intended to be illustrative, and not restrictive. It is intended to cover all alternatives, modifications and equivalents as may be included within the spirit and scope of the invention as defined in the appended claims. Many other embodiments will be apparent to those of skill in the art upon reviewing the above description. The scope of the invention should, therefore, be determined with reference to the appended claims, along with the full scope of equivalents to which such claims are entitled. In the appended claims, the terms ‘including and ‘in which are used as the plain-English equivalents of the respective terms ‘comprising and ‘wherein, respectively.

1. A sensor chip for selective detection of acetone, the sensor chip comprising:
a first layer having a nano-porous silicon fabricated on a P-type Si <100> substrate;
a second layer having molybdenum trioxide (MoO₃) in contact with the first layer; and
a third layer having chrome gold inter digitated electrodes in contact with the second layer.

2. The sensor chip as claimed in claim 1, wherein the nano-porous silicon is fabricated using an anodization technique on the P-type Si <100> substrate with resistivity of 1-10 Ω-cm in an electrolyte solution of HF:C₂H₅OH in a 1:1 ratio using an electrochemical cell.

3. The sensor chip as claimed in claim 1, wherein the nano-porous silicon is fabricated at a current density of 50 mA cm⁻² etching for a duration of 15 minutes and the etched substrate is used as a platform for MoO₃ nanostructures growth.

4. The sensor chip as claimed in claim 1, wherein the first layer comprising nano-porous silicon has silicon nanowires.

5. The sensor chip as claimed in claim 4, wherein the silicon nanowires are formed by depositing a gold film on a silicon wafer and annealing to form gold nanodots on the wafer.

6. The sensor chip as claimed in claim 1, wherein the second layer having molybdenum trioxide is formed by deposition of a molybdenum thin film on the substrate by RF sputtering and oxidization at 500° C. for 60 minutes in order to get molybdenum trioxide (MoO₃).

7. The sensor chip as claimed in claim 1, wherein the chrome-gold is deposited over the molybdenum trioxide (MoO₃) using RF sputtering and metal mask for formation of electrodes on the surface.

8. The sensor chip as claimed in claim 1, wherein the chrome and gold deposition is done using RF sputtering at 300 W power and a gas pressure of 20 mtorr and 10 mtorr respectively.

9. The sensor chip as claimed in claim 1, wherein the first layer has a thickness of 500 nm to 10 microns.

10. The sensor chip as claimed in claim 9, wherein the first layer has a thickness of 1-5 microns.

11. The sensor chip as claimed in claim 1, wherein the second layer has a thickness ranging from 2 nm to 30 nm.

12. The sensor chip as claimed in claim 1, wherein the second layer has a thickness of 10 nm.

13. The sensor chip as claimed in claim 1, wherein the sensor chip is wrapped into a sensor header and integrated with a microcontroller to provide a handheld device.

14. The sensor chip as claimed in claim 1, wherein the chip selectively detects acetone from 0.5 ppm to 100 ppm.

15. The sensor chip as claimed in claim 1, wherein the chip selectively detects acetone over isopropanol and/or ethanol in the presence of 90% relative humidity.

16. A method for fabricating a sensor chip for selective detection of acetone at room temperature, the method comprising:
a) anodizing a P-type Si <100> substrate in an electrolytic solution having hydrofluoric acid and ethanol;
b) fabricating silicon nanowires by metal assisted chemical etching of silicon in hydrofluoric acid solution;
c) providing a MoO₃ thin film on the substrate of step (a) by depositing a molybdenum thin film on the substrate of step (a) by radio frequency reactive sputtering;
d) oxidizing the molybdenum thin film; and
e) depositing inter digitated electrodes (IDEs) on the MoO₃ film by a shadow mask in sputtering.

17. The method as claimed in claim 16, wherein the ratio of hydrofluoric acid:
ethanol in step (a) is 1:1 and the silicon substrate is etched with said hydrofluoric acid ethanol mixture for a period ranging from 30 seconds to 60 minutes.

18. A breath analyzer device for detecting acetone comprising:
a sensor chip for selective detection of acetone at room temperature, wherein the sensor chip comprises:
a first layer having a nano-porous silicon fabricated on a P-type Si <100> substrate;
a second layer having molybdenum trioxide (MoO₃) in contact with the first layer;
a third layer having chrome gold inter digitated electrodes in contact with the second layer; and
a sensor header and an integrated microcontroller.

19. The device as claimed in claim 18, wherein the molybdenum trioxide is fabricated using oxidation of molybdenum at 500° C. for 60 minutes.
